(19) United States
(12) Patent Application Publication
(10) Pub. No.: US 2026/0236050 A1
(43) Pub. Date: Aug. 13, 2026

(54) DRAM-STYLE BIDIRECTIONAL CURRENT INJECTOR AND MULTI-ELECTRODE APPARATUS WITH DRAM-STYLE BIDIRECTIONAL CURRENT INJECTORS

(71) Applicant: President and Fellows of Harvard College, Cambridge, MA (US)

(72) Inventors: Donhee Ham, Cambridge, MA (US); Jun Wang, Cambridge, MA (US); Yuchang Zhang, Cambridge, MA (US)

(73) Assignee: President and Fellows of Harvard College, Cambridge, MA (US)

(21) Appl. No.: 19/151,845

(22) PCT Filed: Jan. 26, 2024
(86) PCT No.: PCT/US2024/013025

§ 371 Date: Jul. 29, 2025

Related U.S. Application Data
(60) Provisional application No. 63/482,269, filed on Jan. 30, 2023.

(51) Int. Cl. G05F 1/56; A61B 5/293; A61B 5/383; H03K 17/687

(52) U.S. Cl. CPC G05F 1/56; A61B 5/293; A61B 5/383; H03K 17/6871; A61B 2562/028; A61B 2562/04

(57) ABSTRACT

A voltage-controlled bi-directional current injector includes a capacitor configured to store an analog voltage provided by a voltage source; base-current circuitry configured to receive the analog voltage from the capacitor and to produce a base current from the analog voltage; a first circuitry branch configured to mirror the base current to produce a first current having a first direction and a magnitude based on the base current; a second circuitry branch configured to mirror the base current to produce a second current having a second direction and a magnitude based on the base current, the second direction being opposite the first direction; and output circuitry configured to allow the first current or the second current to be output as an output current. The analog voltage is provided from the capacitor to the base-current circuitry without use of a clock-signal-controlled switch. The first and second currents may have a same magnitude.

### CROSS REFERENCE TO RELATED APPLICATIONS

The present application claims the benefit of priority of U.S. Provisional Application No. 63/482,269 filed Jan. 30, 2023, entitled “DRAM-STYLE BIDIRECTIONAL CURRENT INJECTOR AND MULTI-ELECTRODE APPARATUS WITH DRAM-STYLE BIDIRECTIONAL CURRENT INJECTORS,” the entire contents of which is incorporated by reference herein.

### FIELD OF THE INVENTION

The present disclosure generally relates to apparatuses and methods that allow an electrode to be stimulated with a current from a bidirectional current injector based on a stored voltage for the electrode. The bidirectional current injector may be one of a plurality of current injectors of a multi-electrode apparatus, with each current injector being individually programmable to store a voltage that may be different from voltages stored by the other current injectors of the apparatus. The apparatus may be used to stimulate a plurality of electrodes with currents having different amplitudes and either of two directions.

### BACKGROUND

Arrays of electrodes have become increasingly popular for a wide variety of applications, such as for conducting electrochemical reactions to screen for biological activity, to prepare chemical libraries, and for electrophysiological stimulation of biological organisms (e.g., animal organs, plants, microorganisms, etc.) and sensing of responses from the organisms, to name a few. For example, multi-electrode arrays (MEAs) have been used for neurological tests to investigate biological responses to various experimental conditions, in vivo as well as in vitro.

In some cases, MEAs based on complementary metal-oxide-semiconductor (CMOS) technology have been found to be particularly suitable for electrical stimulation and sensing of biological organisms due to their ability to be fabricated on semiconductor chips using known microfabrication technologies, thus permitting MEAs to have a wide range of sizes, from dimensions in the submicron range and even in the nanometer range to dimensions that may be limited predominantly by dimensions of wafers from which the chips are cut. Using such technologies, MEAs may be produced to have a high degree of integration and to coexist with other on-chip circuitry. That is, a MEA and circuitry for addressing electrodes of the MEA may be located on a single chip. An amount of chip area occupied by the on-chip circuitry, however, may limit a total number of electrodes on the chip.

### SUMMARY OF THE DISCLOSURE

Aspects of the technology disclosed herein relate to a new approach for stimulating electrodes of a multi-electrode apparatus. In some aspects, the new approach may include circuitry that allows a large number of electrodes to be stimulated with individualized currents. That is, an electrode may be stimulated with an injection current that is different from other injection currents used to stimulate other electrodes of the apparatus. The injection currents may be voltage-controlled currents, and the circuitry may be programmed with desired voltages that are used to generate the individualized bidirectional currents for the electrodes. In some implementations of the present technology, the electrodes may number in the thousands, or tens of thousands, or hundreds of thousands, or over a million, and the circuitry may permit an electrode to be stimulated with a known current based on a programmed voltage for the electrode, which may be different from other programmed voltages for other electrodes. The stimulation currents may vary from electrode to electrode over a wide range. For example, the technology disclosed herein may be utilized to stimulate a first group of one or more electrodes with a relatively small current, e.g., ~1×10⁻⁸ A (~10 pA), while a second group of one or more electrodes is stimulated with a relatively large current, e.g., ~1×10⁻⁶ A (~1 uA), while a third group of one or more electrodes is stimulated with a mid-range current, e.g., ~1×10⁻⁷ A (~0.1 uA).

In some aspects of the technology disclosed herein, the circuitry of the multi-electrode apparatus may permit each of the electrodes to be sensed before and/or during and/or after stimulation. Stimulation of the electrodes may take place without the use of clock signals to control opening and/or closing of switches. Such absence of clock signals may be advantageous as it may reduce an amount of noise present during sensing of the electrodes while stimulation is occurring. For example, high-frequency switching noise, which may occur when a switched capacitor is used to generate a stimulation current, may not be present or may be minimized by the circuitry of the present technology due to the absence of clock signals during sensing of signals from the electrodes.

The circuitry of the multi-electrode apparatus may advantageously permit a larger number of electrodes. That is, the circuitry may have a relatively small “footprint” and therefore may allow a greater number of electrodes to be arranged on the same chip with the circuitry. For example, in contrast to an apparatus that uses switched capacitors to store voltages, such as in an apparatus that uses a binary-weighted current source, which may require clock-controlled transistors to control outputting of the voltage for current generation for each capacitor, the apparatus according to some embodiments of the present technology may storage voltages in capacitors that are not switched capacitors. Thus, the chip space that would have been occupied by clock-controlled transistors and related components may instead be used for more electrodes.

According to an aspect of the technology of the present disclosure, a voltage-controlled bi-directional current injector may be comprised of a capacitor configured to store an analog voltage provided by a voltage source; base-current circuitry configured to receive the analog voltage from the capacitor and to produce a base current from the analog voltage; a first circuitry branch configured to mirror the base current to produce a first current having a first direction and a magnitude based on the base current; a second circuitry branch configured to mirror the base current to produce a second current having a second direction and a magnitude based on the base current, the second direction being opposite the first direction; and output circuitry configured to allow the first current or the second current to be output as an output current. The analog voltage may be provided from the capacitor to the base-current circuitry without use of a clock-signal-controlled switch. The first and second currents may have a same magnitude.

According to some embodiments of this aspect, the first and second circuitry branches may be arranged in parallel to each other.

According to some embodiments of this aspect, the base current may be provided to the first and second circuitry branches without use a clock-signal-controlled switch between the capacitor and the first circuitry branch and without use of a clock-signal-controlled switch between the capacitor and the second circuitry branch.

According to some embodiments of this aspect, the first circuitry branch may include a first current mirror comprising a plurality of a first type of MOS transistor, and the second circuitry branch may include a second current mirror comprising a plurality of a second type of MOS transistor. In some embodiments, the first circuitry branch may include a plurality of PMOS transistors configured as a first current mirror, and the second circuitry branch may include a plurality of NMOS transistors configured as a second current mirror.

In some embodiments, the first current mirror or the second current mirror may be a cascode current mirror. In some embodiments, each of the first and second current mirrors may be a cascode current mirror.

According to some embodiments of this aspect, the output circuitry may include a first output switch located on an output side of the first circuitry branch, and a second output switch located on an output side of the second circuitry branch.

According to some embodiments of this aspect, the analog voltage stored by the capacitor may have a range from VSS to VDD. In some embodiments, VSS may be about 0 V (e.g., ground potential) and VDD may be about 3.3 V. In some embodiments, an absolute value of the output current may be in a range of about 10 pA to about 1 mA. In some embodiments, the absolute value of the output current may be in a range of about 10 pA to about 100 μA. In some embodiments, the absolute value of the output current may be in a range of about 10 pA to about 10 μA.

According to some embodiments of this aspect, the current injector may further be comprised of an input switch configured to control a connection of the capacitor to the voltage source. The input switch may be in a closed state when the capacitor is being addressed by the voltage source to receive the analog voltage, and the input switch may be in an opened state when the capacitor is not being addressed by the voltage source.

According to another aspect of the present technology, a multi-electrode apparatus may be comprised of a voltage generator; a plurality of electrodes; and a plurality of bi-directional current injectors configured to provide output currents to the electrodes. Each of the current injectors may be comprised of a capacitor configured to store an analog voltage provided by the voltage generator, base-current circuitry configured to receive the analog voltage from the capacitor and to produce a base current from the analog voltage, a first circuitry branch configured to mirror the base current to produce a first current having a first direction and a magnitude based on the base current, a second circuitry branch configured to mirror the base current to produce a second current having a second direction and a magnitude based on the base current, the second direction being opposite the first direction, and output circuitry configured to allow the first current or the second current to be output as an output current. For each of the current injectors, the analog voltage may be provided from the capacitor to the base-current circuitry without use of a clock-signal-controlled switch. The first and second currents may have a same magnitude.

According to various embodiments of this aspect, for each of the current injectors, the first and second circuitry branches may be arranged in parallel to each other.

According to various embodiments of this aspect, for each of the current injectors, the base current may be provided to the first and second circuitry branches without use of a clock-signal-controlled switch between the capacitor and the first circuitry branch and without use of a clock-signal-controlled switch between the capacitor and the second circuitry branch.

According to various embodiments of this aspect, for each of the current injectors, the first circuitry branch may include a first current mirror comprising a plurality of a first type of MOS transistor, and the second circuitry branch may include a second current mirror comprising a plurality of a second type of MOS transistor.

According to various embodiments of this aspect, for each of the current injectors, the first circuitry branch may include a plurality of PMOS transistors configured as a first current mirror, and the second circuitry branch may include a plurality of NMOS transistors configured as a second current mirror.

According to various embodiments of this aspect, for each of the current injectors, the first current mirror or the second current mirror may be a cascode current mirror.

According to various embodiments of this aspect, for each of the current injectors, each of the first and second current mirrors may be a cascode current mirror.

According to various embodiments of this aspect, for each of the current injectors, the output circuitry may include a first output switch located on an output side of the first circuitry branch, and a second output switch located on an output side of the second circuitry branch.

According to various embodiments of this aspect, for each of the current injectors, the analog voltage stored by the capacitor may be in a range from VSS to VDD. In some embodiments, VSS may be about 0 V (e.g., ground potential) and VDD may be about 3.3 V. In some embodiments, for each of the current injectors, an absolute value of the output current may be in a range of about 10 pA to about 1 mA. In some embodiments, for each of the current injectors, the absolute value of the output current is in a range of about 10 pA to about 100 μA. In some embodiments, for each of the current injectors, the absolute value of the output current may be in a range of about 10 pA to about 10 μA.

According to various embodiments of this aspect, the electrodes and the current injectors may be disposed on a single substrate. In some embodiments, the single substrate may be a single semiconductor chip.

According to various embodiments of this aspect, each of the current injectors may further be comprised of an input switch configured to control a connection of the capacitor to the voltage generator. The input switch may be in a closed state when the capacitor is being addressed by the voltage generator to receive the analog voltage, and the input switch may be in an opened state when the capacitor is not being addressed by the voltage generator.

According to various embodiments of this aspect, the voltage generator may be configured to address the current injectors individually to provide individual analog voltages to the current injectors. In some embodiments, the individual analog voltages provided by the voltage generator to the current injectors may be different from each other. In some embodiments, at least two of the current injectors may be individually provided with a same analog voltage.

According to various embodiments of this aspect, the apparatus may be further comprised of a controller configured to control values of the individual analog voltages provided by the voltage generator to the current injectors and to cause the input switches of the current injectors to close individually to receive the individual analog voltages from the voltage generator. In some embodiments, the controller may be provided with a clock signal to synchronize delivery of the individual analog voltages to the current injectors, such that a value of the analog voltage is known for each of the current injectors. In some embodiments, the electrodes may be arranged in an array of columns and rows, and the controller may cause the input switches of the current injectors to close sequentially from a first one of the electrodes to a last one of the electrodes.

According to various embodiments of this aspect, a total number of the electrodes may be in a range of 2000 to 1,000,000, or in a range of 20,000 to 10,000, or in a range of 10,000 to 100,000, or in a range of 100,000 to 500,000, or in a range of 500,000 to 1,00,000.

According to various embodiments of this aspect, the electrodes may comprise some of or all of a microelectrode array (MEA) and may be configured to perform sensing and/or stimulation of an object.

According to another aspect of the present technology, a method of using a voltage-controlled bi-directional current injector may be comprised of storing, in a capacitor, an analog voltage provided by a voltage source; providing, by the capacitor, the analog voltage to base-current circuitry to produce a base current from the analog voltage; producing, by a first circuitry branch, a first current from the base current, the first current having a first direction and a magnitude based on the base current; producing, by a second circuitry branch, a second current from the base current, the second current having a magnitude based on the base current first and having a second direction opposite the first direction; and outputting the first current or the second current as an output current. The analog voltage may be provided from the capacitor to the base-current circuitry without use of a clock-signal-controlled switch. The first and second currents may have a same magnitude.

According to various embodiments of this aspect, the first circuitry branch may include a first current mirror comprising a plurality of a first type of MOS transistor, and the second circuitry branch may include a second current mirror comprising a plurality of a second type of MOS transistor. In some embodiments, the first circuitry branch may include a plurality of PMOS transistors configured as a first current mirror, and the second circuitry branch may include a plurality of NMOS transistors configured as a second current mirror.

In some embodiments, the first current mirror or the second current mirror may be a cascode current mirror. In some embodiments, each of the first and second current mirrors may be a cascode current mirror.

According to another aspect of the present technology, a method of using a multi-electrode apparatus may be comprised of providing, by a voltage generator, a plurality of analog voltages to a plurality bi-directional current injectors; at each of the current injectors: storing, in a capacitor of the current injector, an analog voltage received from the voltage generator, providing the analog voltage to base-current circuitry of the current injector to produce a base current from the analog voltage, providing the base current to first and second circuitry branches of the current injector, producing, by the first circuitry branch, a first current having a first direction and a magnitude based on the base current, producing, by the second circuitry branch, a second current having a second direction and a magnitude based on the base current, the second direction being opposite the first direction, and outputting the first current or the second current as an output current of the current injector; and providing the output currents from the current injectors to a plurality of electrodes. At each of the current injectors, the analog voltage may be provided from the capacitor to the base-current circuitry without use of a clock-signal-controlled switch. The first and second currents may have a same magnitude.

According to various embodiments of this aspect, the providing of the analog voltages by the voltage generator may be comprised of addressing the current injectors individually.

According to various embodiments of this aspect, at each of the current injectors, the providing of the base current to the first and second circuitry branches may occur without use of a clock-signal-controlled switch between the capacitor and the first circuitry branch and without use of a clock-signal-controlled switch between the capacitor and the second circuitry branch.

According to various embodiments of this aspect, the providing of the analog voltages by the voltage generator may be comprised of controlling the voltage generator to generate the analog voltages to have different values for different ones of the current injectors, and controlling input switches of the current injectors to open individually such that, for each of the current injectors, a value of the analog voltage provided to the current injector is predetermined for the current injector. In some embodiments, the analog voltages provided by the voltage generator to the current injectors may be different from each other. In some embodiments, at least two of the current injectors may be individually provided with a same analog voltage. In some embodiments, the providing of the analog voltages by the voltage generator may be comprised of controlling the input switches of the current injectors to open sequentially.

According to various embodiments of this aspect, the first circuitry branch may include a plurality of transistors configured as a first current mirror, and the second circuitry branch may include a plurality of transistors configured as a second current mirror. In some embodiments, the first current mirror or the second current mirror may be a cascode current mirror. In some embodiments, each of the first and second current mirrors may be a cascode current mirror.

### BRIEF DESCRIPTION OF THE DRAWINGS

A skilled artisan will understand that the accompanying drawings are for illustration purposes only. It is to be understood that in some instances various aspects of the present invention may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the claims or the present disclosure in any way. In the drawings:

FIG. 1 shows a block diagram of a multi-electrode apparatus, according to some embodiments of the present technology;

FIG. 2 schematically depicts a DRAM-style current injector with parallel branches, according to some embodiments of the present technology;

FIG. 3A schematically depicts a state of output switches for outputting a positive stimulation current, according to some embodiments of the present technology;

FIG. 3B schematically depicts a state of output switches for outputting a negative stimulation current, according to some embodiments of the present technology;

FIG. 4A schematically depicts a first type of base-current generator, according to some embodiments of the present technology;

FIG. 4B schematically depicts a second type of base-current generator, according to some embodiments of the present technology;

FIG. 5 schematically depicts a voltage source configured to program a plurality of current injectors, according to some embodiments of the present technology;

FIG. 6 shows a chart of generated current as a function of voltage used to produce the generated current.

### DETAILED DESCRIPTION

A multi-electrode apparatus according to some embodiments of the present technology, which is able to perform multiple experiments concurrently, may be beneficial for investigations where optimal parameters for a desired reaction are not known for certain experimental conditions. For example, it may be desirable to be able to stimulate a plurality of electrodes at a plurality of different currents to determine an optimal current for achieving a desired biological reaction under certain environmental conditions (e.g., a temperature and/or a pressure at which the biological reaction is being performed and/or a chemical environment in which the biological reaction is being performed, etc.), or for driving a desired electrochemical reaction under certain environmental conditions (e.g., a pH of fluid in which the electrochemical reaction is occurring and/or a concentration of one or more reactants in the gaseous or liquid environment in which the electrochemical reaction is occurring, etc.).

To minimize variations in conditions that may occur from investigation to investigation, it may be beneficial to perform as many experiments as possible in parallel on a single chip. In addition to parallel experiments, for statistically reliable assessments, it may be desirable for some of the parallel experiments to be duplicates of each other. For example, it may be desirable to have a first group of two or more electrodes on a chip perform a first experiment at a first stimulation current, a second group of two or more electrodes on the chip perform a second experiment at a second stimulation current, a third group of two or more electrodes on the chip perform a third experiment at a third stimulation current, and so on. The first, second, and third experiments may be performed concurrently so that the environmental conditions of all the electrodes on the chip, other than their stimulation currents, may be the same or nearly the same. As will be appreciated, however, the number of experiments that may be performed on a single chip may be limited by the on-chip circuitry used to control the experiments. That is, the area of the chip occupied by the on-chip circuitry may determine a maximum number of electrodes on the chip. Moreover, as the complexity of the experiments increase, the number of components of the on-chip circuitry and the complexity of the components may increase. For example, the chip may comprise a plurality of temperature sensors located on various parts of the chip to monitor local temperatures during an experiment, which may require area for wiring in addition to the area taken up by the sensors. This may further reduce the available area on the chip for electrodes.

The technology disclosed herein may be used advantageously to simplify the on-chip circuitry of a multi-electrode apparatus while providing enhanced flexibility to stimulate a plurality of electrodes individually, such that the electrodes are stimulated with a plurality of different currents of either direction. The disclosed technology may, in a sense, bring to mind a dynamic random-access memory (DRAM) device in that the technology utilizes a capacitor as a non-volatile storage device to store a voltage from which an injection current is generated. The term “injection current” may be used interchangeably with the term “stimulation current” herein to refer to a current provided to an electrode to stimulate the electrode. As described herein, the apparatus may comprise a plurality of current injectors. Each current injector of the apparatus may be individually programmed with a voltage used to generate a base current, which may be mirrored via mirror circuitry of the current injector to produce first and second currents having a same amplitude and opposite polarities, i.e., a positive current and a negative current having equal amplitudes. The mirror circuitry may be comprised of parallel branches configured to produce the first and second currents. The first and second currents may be referred to herein as bidirectional currents. According to some embodiments of the present technology, based on configuration bits stored in an on-chip configuration memory of the apparatus, the positive current or the negative current may be provided to the electrode, thus eliminating the need for an on-chip clock to provide clock signals for controlling the opening and closing of switches used to generate and output a desired stimulation signal to the electrode. In other words, the capacitors in the current injectors are not switched capacitors and therefore the current injectors do not require clock signals to control the opening of closing of switches in order to generate and output stimulation currents to the electrodes.

A beneficial aspect of not using switched capacitors is that switching noise, which typically would result from clock-controlled switching operations (e.g., high-frequency noise resulting from opening and closing of switches to permit stored voltages in the capacitors to be used to generate currents), would be eliminated. Such noise reduction may advantageously increase a signal-to-noise ratio (SNR) of signals sensed from the electrodes. For example, the apparatus may be configured such that the electrodes may each be stimulated with a stimulation current and concurrently may each be sensed to determine voltages (e.g., open-circuit voltages (OCVs)) at the electrodes during stimulation. For some investigations, the OCVs may be small and may have noise values that mask the OCVs. The elimination of clock-generated switching noise may permit the OCVs to be more readily discerned from circuit noise and/or other noise that may be present in signals sensed from the electrodes, thus permitting weak or low-amplitude signals to be detected.

The circuitry of the multi-electrode apparatus may advantageously permit a larger number of electrodes. That is, the circuitry may have a relatively small “footprint” and therefore may allow a greater number of electrodes to be arranged on the same chip with the circuitry. For example, in contrast to an apparatus that uses switched capacitors, which requires continuous clocks to switch capacitors for current generation for each current injector, the apparatus according to some embodiments of the present technology may store voltages in capacitors that are not switched capacitors. Thus, the chip space that would have been occupied by clock-controlled transistors may be used instead for more electrodes. In some embodiments, the capacitors of the apparatus may be easily programmed using a single on-chip voltage generator or a small number of on-chip voltage generators (e.g., ten or fewer voltage generators). Such an advantage may result from a simplified layout design that is possible with the circuitry of the apparatus, thus permitting high-throughput programming of the capacitors. For example, a voltage generator with thousands of programming channels each corresponding to an electrode may be arranged in a simple layout design on the chip. As will be appreciated, for a relatively small number of capacitors (e.g., tens of thousands or fewer), the time required for programming or storing the voltages in the capacitors may not be a concern, even if only a single voltage generator is used. On the other hand, for a relatively large number of capacitors (e.g., hundreds of thousands or greater), programming time may be a significant concern, especially if only a small number of voltage generators are used. The high-throughput programming permitted by some embodiments of the present technology may advantageously reduce the number of on-chip voltage generators needed for programming the capacitors, thus freeing up space for additional electrodes. The simple layout design permitted by some embodiments of the present technology may advantageously simplify scaling up of the apparatus to use larger and larger chips with more and more electrodes per chip.

According to some embodiments of the present technology, prior to starting a process in which electrodes of a multi-electrode apparatus are stimulated by currents produced by current injectors of the apparatus, capacitors of the current injectors may be addressed one by one such that the capacitors may each store a voltage designated to be used by a corresponding current injector to generate bidirectional stimulation currents, i.e., a positive current and a negative current. In some embodiments, although two currents may be generated, one of the two currents may be provided to a corresponding electrode according to the configuration bits stored for that electrode. The addressing of the capacitors of the current injectors may occur one by one or in groups of two or more, and may be controlled using clock signals to enable access to individual ones of the current injectors. For example, for an array of current injectors arranged in columns and rows, input switches in the current injectors may be controlled to open selectively using clock signals, thus permitting programming of the capacitors via, e.g., rastering sequentially across rows and from column to column, thus allowing a desired voltage to be provided to each of the capacitors by one or more voltage generators. The desired voltages may be different from each other for some of or all of the capacitors, or may be the same as each other for all of the capacitors. For example, the capacitors may include a first group of one or more capacitors programmed to store a first voltage, a second group of one or more capacitors programmed to store a second voltage, a third group of one or more capacitors programmed to store a third voltage, etc. In another example, for experiments where a plurality of capacitors are to be programmed to store the same voltage, input switches corresponding to those capacitors may be controlled to be open concurrently, thus allowing simultaneous programming of those capacitors to store the same voltage provided by one or more voltage generators, which may advantageously reduce programming time. Although the one or more voltage generators are described to be on-chip, in some embodiments, the capacitors of the apparatus may be programmed by one or more voltage generators located off-chip. In such embodiments, the on-chip circuitry may not include a voltage generator thus freeing up space for additional electrodes.

According to some embodiments of the present technology, each current injector of the multi-electrode apparatus may output a stimulation current to a single electrode corresponding to that current injector. In some other embodiments, one or more current injectors of the apparatus may each output a stimulation current to more than one electrode.

According to some embodiments of the present technology, once the capacitors of the current injectors have been programmed to store respective voltages for generating bidirectional stimulation currents, the process may commence without requiring the use of clock signals to access the stored voltages. Because use of the clock signals takes place before stimulation of the electrodes and before signals are sensed from the electrodes, noise that may associated with the clock signals (e.g., high-frequency switching noise) may not be present and therefore the sensed signals may not be masked by the noise, thus permitting a higher SNR than processes during which clock signals occur while the electrodes are being sensed. In some embodiments, the on-chip circuitry of the multi-chip apparatus may include one or more clock-signal generators, which may be used during programming of the capacitors, as described herein, but which may be inactive during an experimental process in which signals are being sensed from the electrodes.

According to some embodiments of the present technology, each current injector may be configured such that a base branch or section of the current injector generates a base current from the voltage stored in the capacitor. The voltage-generated base current may be provided to positive and negative branches of sections of the current injector to generate bidirectional currents, which have the same amplitude and opposite polarities. In some embodiments, the positive and negative branches may be configured with circuitry to mirror the base current, such that the bidirectional currents are generated to have the same amplitude as the base current, with one being a positive current and one being a negative current. The current injector may output the positive current or the negative current, which may be provided as a stimulation current to at least one of the electrodes of the apparatus. Whether the positive current or the negative current is output as the stimulation current may be determined according to the configuration bits stored in the on-chip configuration memory.

For some investigations, a high degree of integration may be beneficial, so that stimulation and/or sensing of an object over very short distances may take place (e.g., sub-mm distances and even distances on the order of hundreds of μm or smaller). For example, a high-density MEA in which a large number of electrodes are closely spaced relative to each other may permit fine variations in stimulation currents to be provided to the object (e.g., a part of an animal, a part of a plant, etc.), which may permit an investigation of how various levels of stimulation may affect the object. For some investigations, such as those in which an object is to be stimulated over a relatively large area (e.g., several mm to a few cm or more), having the electrodes span as much of the on-chip area as possible may be desirable, so that more of the object may be sensed concurrently. Various embodiments of the present technology may permit MEAs to be used without on-chip circuitry for clocks and clock-signal-controlled switches for accessing the voltages stored in the capacitors, thus increasing the on-chip area useable for electrodes.

According to some embodiments of the present technology, a DRAM-style current injector (DCI) may use a capacitor to store an analog voltage that determines an amplitude of an injection current for stimulating one or more electrodes of a multi-electrode apparatus. A DCI is analogous to a DRAM cell in that both are device components configured to store quantities to be used by the devices in which they are deployed. For example, a DRAM cell and an DCI both use a capacitor to store a charge used to control a gate voltage of a transistor. However, whereas a DRAM cell may use a capacitor to store a binary bit to be used by a digital device, a DCI may use a capacitor to store an analog voltage used to control an amplitude of a current produced by the DCI.

The analog voltage may be used to generate a voltage-controlled current having a desired value. That is, use of an analog voltage permits the current produced by the DCI to have a desired amplitude without limitations that would be caused by using a digital voltage that can be varied only by discrete amounts. The voltage-controlled current is provided to a pair of current mirrors (e.g., PMOS and NMOS current mirrors) that generate a positive current and a negative current, one of which is injected to the one or more electrodes for stimulating the one or more electrodes. The multi-electrode apparatus may include a plurality of DCIs. In some embodiments, each electrode of the apparatus may be associated with its own DCI, such that the capacitor of the DCI may a custom voltage for the electrode, which may be different from and independent of the voltages stored in the capacitors of other DCIs of the apparatus. Such customization may be used advantageously to customize how an object is to be stimulated. In one example, the DCIs may be programmed with customized voltages designed to result in a gradient of stimulation currents that may be provided to an area of the object via the electrodes of the apparatus. In another example, a predetermined pattern of positive and negative stimulation currents may be provided to an area of the object. As will be appreciated, the flexibility to program the capacitors of the DCIs permits numerous possibilities for stimulating the object.

In addition to each DCI advantageously being able to be addressed individually and programmed to inject a current independently of other DCIs, according to embodiments of the present technology, another advantage of DCIs of the reduction in chip area or real estate when DCIs are used compared to stimulation schemes that utilize a binary-weighted current source. By using an analog voltage to drive current mirrors to produce bidirectional currents, a DCI may be used to produce a stimulation current having an arbitrarily small amplitude, or an arbitrarily large amplitude, or anything in between, with a value that depends on the voltage programmed or stored in the capacitor of the DCI. In some embodiments, the wide range of amplitudes for the stimulation current may span several orders of magnitudes, e.g., from ~10 pA to ~1 uA, due to exponential current-voltage (IV) characteristics typical of MOSFETS used for generating current from voltage.

A further advantage of DCIs, according to some embodiments of the present technology, is that DCIs may be arranged in a simple array that permits easy programming of the capacitors. Such simplicity may be particularly useful for designing a high throughput programming routine for programming, e.g., hundreds of thousands of DCIs or more via thousands of on-chip communication channels to the capacitors of the DCIs. Each DCI may have a relatively compact layout area, which may be easily arranged into of a desired shape and size, and also may be easily scaled up for chips of larger and larger areas.

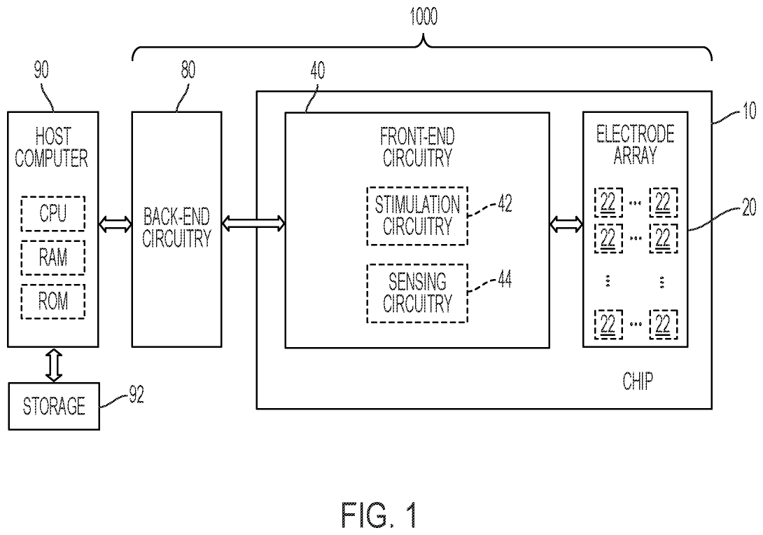

Turning now to the drawings, FIG. 1 schematically shows a multi-electrode apparatus 1000, according to some embodiments of the present technology. The apparatus 1000 may include an electrode array 20 comprised of a plurality of electrodes 22 in communication with and operably connected to front-end circuitry 40. In some embodiments, CMOS technology may be used to fabricate the electrodes 22 and the front-end circuitry 40 on a single semiconductor chip 10. The front-end circuitry 40 may include stimulation circuitry 42 and sensing circuitry 44. The stimulation circuitry may be configured to provide the electrodes 22 with a stimulation signal. The sensing circuitry 44 may be configured sense signals from the electrodes 22 before and/or during and/or after stimulation of the electrodes. The front-end circuitry 40 also may include other on-chip circuitry (not shown). For example, the front-end circuitry 40 may include any one or any combination of: voltage source(s), amplifier(s), buffer(s), shift register(s), multiplexer(s), memory(ies), clock(s), switch(es), heater(s), temperature sensor(s), analog-to-digital converter(s) (ADC(s)), and the like. In some embodiments, the stimulation circuitry 42 may be comprised of a plurality of DRAM-style current injectors, discussed in more detail below.

According to some embodiments of the present technology, the apparatus 1000 also may include back-end circuitry 80 in communication with and operably connected to the front-end circuitry 40. The back-end circuitry 80 may be located off-chip and may be connected to the front-end circuitry 40 via, e.g., wire bonds. In some embodiments, contact pads on the chip 10 may be connected with contact pads on a substrate (e.g., a PCB) on which the back-end circuitry 80 is disposed. In some embodiments, the back-end circuitry 80 may include any one or any combination of (not shown): voltage source(s) (e.g., reference voltage source(s), bias voltage source(s), and the like), power supply(ies), FPGA(s), digital-to-analog converter(s) (DAC(s)), ADC(s), input-output interface(s) (e.g., a USB interface), and the like. In some embodiments, the back-end circuitry 80 may be disposed on a PCB to which the chip 10 is mounted. As will be appreciated, other connection techniques may be employed for connecting the chip 10 to the back-end circuitry 80. In some embodiments, a host computer 90 may be in communication with and operably connected to the back-end circuitry 80 and may be configured to communicate signals to and receive signals from the front-end circuitry 40 via the back-end circuitry 80. In some embodiments, the host computer 90 may include at least one microprocessor (e.g., CPU(s)), volatile memory (e.g., RAM), and non-volatile memory (e.g., ROM). A storage device 92 may be operably connected to the host computer 90 and may be configured to record sensed data based on signals sensed from the electrodes 22 and/or to store voltage data for programming the stimulation circuitry 42 and/or store configuration data for the front-end circuitry 40 to stimulate each of the electrodes 22 individually with a desired stimulation signal (e.g., to stimulate a first electrode with a negative current corresponding to a programmed voltage for the first electrode, and to stimulate a second electrode with a positive current corresponding to a programmed voltage for the second electrode, etc.). For example, the host computer 90 may provide configuration data to the front-end circuitry 40 to update configuration bits stored in a configuration memory of the front-end circuitry 40. The configuration bits may be used by the apparatus 1000 to control a stimulation procedure and/or a sensing procedure. The storage device 92 may store computer-readable code executed by the microprocessor(s) to control one or more operations or procedures of the apparatus 1000. For example, the microprocessor(s) may execute the computer-readable code to control a stimulation procedure to stimulate the electrodes 22 and/or to control a sensing procedure to sense signals from the electrodes 22.

According to some embodiments of the present technology, the stimulation circuitry 42 may include a plurality of DRAM-style current injectors configured to stimulate the electrodes 22. In some embodiments, each current injector may be an analog circuit configured to store a voltage and to generate bidirectional currents, one of which is selected provided to a corresponding one of the electrodes 22 as the stimulation current for that electrode 22.

According to some embodiments of the present technology, the electrodes 22 may each form part of a pixel of the apparatus 1000. In some embodiments, each pixel may comprise an electrode 22 and a pixel circuit (not shown) operably connected to the electrode 22. The pixel circuit may be configured to stimulate the electrode 22 (e.g., with a stimulation current) and/or to sense a signal from the electrode 22 (e.g., an OCV). In some embodiments, the electrode array 20 may overlay an array of pixels circuits such that each electrode 22 is positioned above a corresponding pixel circuit, thus forming a pixel array. In some embodiments, some or all of the electrodes 22 may not be located above their corresponding pixel circuits but may instead be located remotely in one or more separate regions on the chip 10. On-chip wiring may connect the electrodes 22 and their remotely located pixel circuits. In some embodiments, each pixel circuit may comprise a DRAM-style current injector, discussed in more detail elsewhere herein.

Although the stimulation circuitry 42 is depicted in FIG. 1 to be a discrete unit of the front-end circuitry 40, it should be understood that the stimulation circuitry 42 may be comprised of a plurality of circuits located at a plurality of locations on the chip 10. The sensing circuitry 44 may be configured to detect or sense electrical signals from the electrodes 22. Although the sensing circuitry 44 is depicted in FIG. 1 to be a discrete unit of the front-end circuitry 40, it should be understood that the sensing circuitry 44 may be comprised of a plurality of circuits located at a plurality of locations on the chip 10. In some embodiments, the front-end circuitry 40 may be comprised of a plurality of circuits each configured to output a stimulation current and to sense an OCV from a corresponding one of the electrodes 22. In some embodiments, the OCV may be sensed during stimulation of the electrode 22. Additional details regarding electrode structures, multi-electrode arrays, pixel circuits, and stimulation and sensing of electrodes in a multi-electrode array may be found in International Patent Application No. PCT/US2022/033228 (Attorney Docket No. H0498.70743WO00), which is incorporated by reference herein in its entirety.

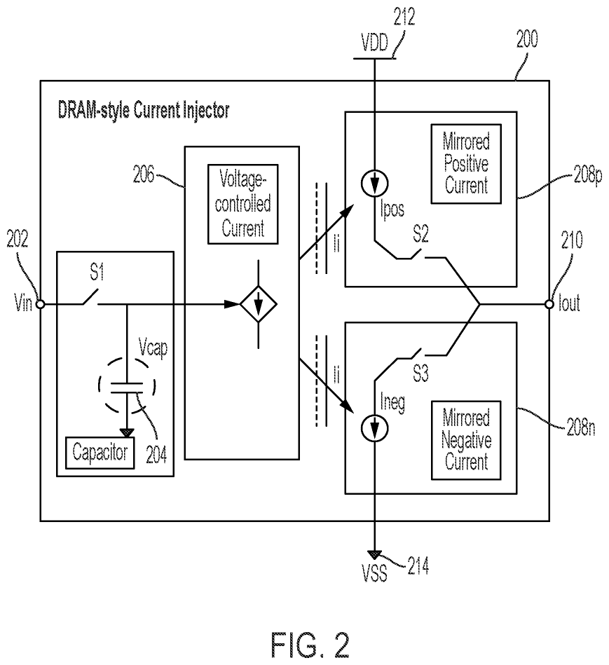

FIG. 2 schematically depicts a DRAM-style current injector 200, according to some embodiments of the present technology. The DCI 200 may comprise an input node 202 through which an input voltage Vᵢₙ may be provided to a capacitor 204 via an input switch S1. During programming of the capacitor 204, the input switch S1 may be controlled to be in a closed state to enable the input voltage Vᵢₙ to reach the capacitor 204. The capacitor 204 may store a programmed voltage V_{cap} corresponding to the input voltage Vᵢₙ. After programming of the capacitor 204, the input switch S1 may be controlled to be in an opened state to prevent the capacitor 204 from receiving an input voltage intended for another capacitor. The input voltage Vᵢₙ may be held in the capacitor 204 until the capacitor is reprogrammed.

According to some embodiments of the present technology, the programmed voltage V_{cap} is provided to a voltage-controlled current generator 206 configured to generate an initial current Iᵢ having a value that is set by the programmed voltage V_{cap}. The initial current Iᵢ may also be referred to herein as the base current Iᵢ. In some embodiments, the programmed voltage V_{cap} may be provided directly to the current generator 206 without an intervening switch. The current generator 206 may generate the base current Iᵢ having a desired magnitude set by the programmed voltage V_{cap}. Bidirectional currents may be produced from the base current Iᵢ. In some embodiments, the base current Iᵢ may be used to produce a positive current and a negative current having the same magnitude as that of the base current Iᵢ. In some embodiments, the base current Iᵢ may be used to produce a positive current and a negative current having magnitudes that are scaled to the magnitude of the base current Iᵢ (e.g., an attenuated magnitude that is less than the magnitude of the base current Iᵢ or an enhanced magnitude that is greater than the magnitude of the base current Iᵢ). The positive and negative currents may have a same magnitude. In some embodiments, the base current Iᵢ may be provided to a first current mirror 208p with circuitry for producing a positive current Iₚₒₛ, i.e., a current having a positive direction. The base current Iᵢ also may be provided to a second current mirror 208n with circuitry for producing a negative current I_{neg}, i.e., a current having a negative direction. In some embodiments, the first and second current mirrors 208p, 208n may be arranged parallel to each other. Output switches S2, S3 may be controlled to permit the positive current Iₚₒₛ or the negative current I_{neg} to be output as a stimulation current Iₒᵤₜ that flows through a corresponding one or more of the electrodes 22. The DCI 200 may have a positive supply connection 212 and a negative supply connection 214. In some embodiments, the positive supply connection 212 may have a voltage V_{DD}, and the negative supply connection 214 may have a voltage V_{SS}. In some embodiments, V_{SS} may be at ground potential, as shown in FIG. 2.

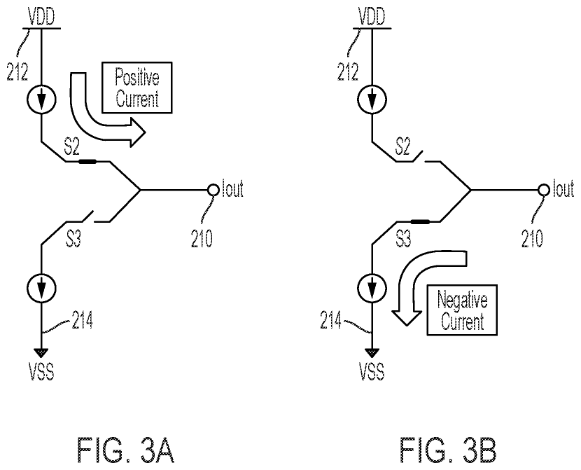

More specifically, as schematically depicted in FIG. 3A, if an experimental process requires the stimulation current to be a positive current having a magnitude corresponding to the base current Iᵢ generated according to the programmed voltage V_{cap}, the output switch S3 may be controlled to be in an opened state, thus preventing the negative current I_{neg} from reaching the electrode(s) 22, and the output switch S2 may be controlled to be in a closed state, thus permitting the positive current Iₚₒₛ to flow in a direction from the positive supply connection 212 to the electrode(s) 22 via an output node 210. On the other hand, as schematically depicted in FIG. 3B, if an experimental process requires the stimulation current to be a negative current having a magnitude corresponding to the base current Iᵢ generated according to the programmed voltage V_{cap}, the output switch S2 may be controlled to be in an opened state, thus preventing the positive current Iₚₒₛ from reaching the electrode(s) 22, and the output switch S3 may be controlled to be in a closed state, thus permitting the negative current I_{neg} to flow from the electrode(s) 22 to the negative supply connection 214 via the output node 210. As indicated above, the negative supply connection 214 may be ground.

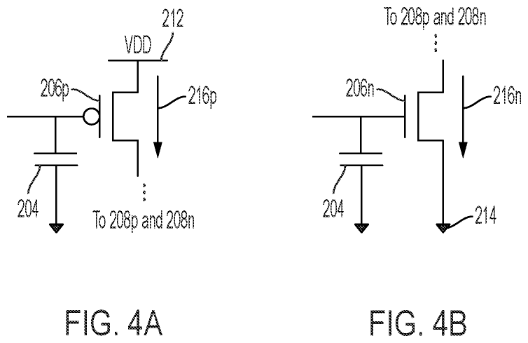

In some embodiments of the present technology, the current generator 206 may have a simple structure comprising a transistor connected to the capacitor 204. In some embodiments, the current generator 206 may be comprised of a PMOS transistor 206p connected between the positive supply connection 212 and the first and second current mirrors 208p, 208n, as schematically depicted in FIG. 4A. An arrow 216p indicates a direction of current flow for the PMOS transistor 206p. In some embodiments, the current generator 206 may be comprised of an NMOS transistor 206n connected between the negative supply connection 214 and the first and second current mirrors 208p, 208n, as schematically depicted in FIG. 4B. An arrow 216n indicates a direction of current flow for the NMOS transistor 206n. As will be appreciated, the first and second current mirrors 208p, 208n may have configurations other than those shown in FIGS. 4A and 4B. In some embodiments, the first current mirror 208p and/or the second current mirror 208n may be a cascode current mirror. It should be understood that although the term “negative” is used for the negative supply connection 214, the negative supply connection may have a potential V_{SS} that is zero or greater than zero but less than V_{DD}

According to some embodiments of the present technology, the apparatus 1000 may include a plurality of the current injectors 200 operably connected to the electrodes 22 to provide individualized stimulation currents to the electrodes 22. That is, a first group of one or more of the electrodes 22 may be stimulated with a first stimulation current and second group of one or more of the electrodes 22 may be stimulated with a second stimulation current different from the first stimulation current. Each of the current injectors 200 may be programmed to store a programmed voltage V_{cap} having a predetermined value for producing a desired base current Iᵢ. In some embodiments, the current injectors 200 may be located remotely from the electrodes 22, e.g., in different regions of the chip 10. The chip may comprise wiring configured to transmit stimulation currents to the electrodes 22 and to transmit signals sensed from the electrodes 22 (e.g., OCVs). The current injectors 200 may be connected to one or more voltage sources configured to provide the input voltages Vin to the capacitors 204. In some embodiments, the voltage source(s) may be comprised of one or more voltage generators configured to output a plurality of different desired voltages individually to the capacitors 204, one by one, or to output a desired voltage to a group of two or more capacitors 204 concurrently.

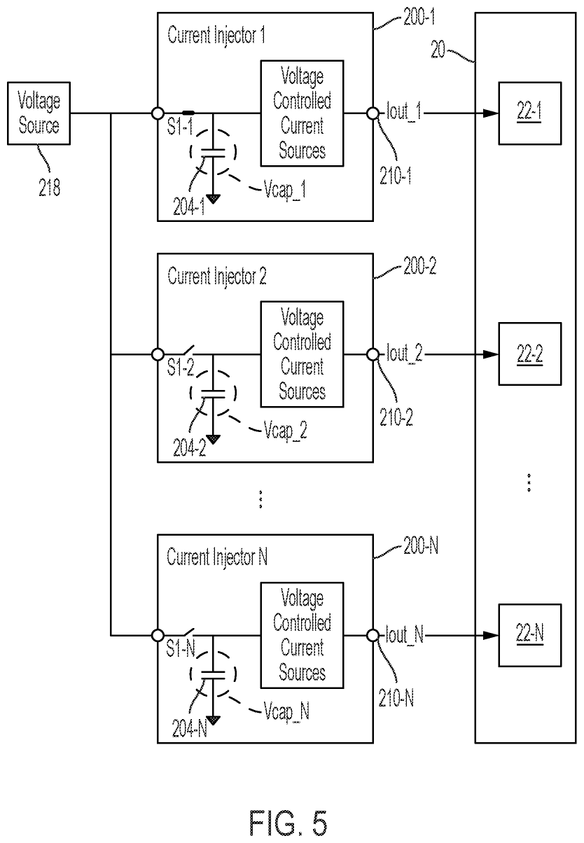

FIG. 5 schematically depicts a voltage source 218 operably connected to N current injectors 200-1, 200-2, . . . , 200-N, where N may have a value of 100 or greater or 1000 or greater or 10,000 or greater or 100,000 or greater. The voltage source 218 may be a voltage generator programmed to output a plurality of different input voltages Vᵢₙ, which may be stored individually in corresponding capacitors 204-1, 204-2, . . . , 204-N as programmed voltages V_{cap_1}, V_{cap_2}, . . . , V_{cap_N}. In some embodiments, during programming of a first current injector 200-1, an input switch S1-1 of the current injector 200-1 may be controlled to be in the closed position, thus permitting transmission of a first voltage from the voltage source 218 to the capacitor 204-1 of the first current injector 200-1. The first voltage may be stored in the capacitor 204-1 as the programmed voltage V_{cap_1} for the first current injector 200-1. During programming of the first current injector 200-1, input switches S1-2, . . . , S1-N of the other current injectors 200-2, . . . , 200-N may be controlled to be in the opened position, thus preventing transmission of the first voltage from the voltage source 218 to the capacitors 204-2, . . . , 204-N of the other current injectors 200-2, . . . , 200-N. In some embodiments, a group of two of more of the current injectors 200-1, 200-2, . . . , 200-N may be programmed concurrently with the first voltage by appropriately controlling the input switches to be closed for each member of the group. Output nodes 210-1, 210-2, . . . , 210-N of the current injectors 200-1, 200-1, . . . , 200-N may each be connected to corresponding electrodes 22-1, 22-2, . . . , 22-N, as depicted in FIG. 5.

According to some embodiments of the present technology, the voltage source 218 may be one of a plurality of voltage sources 218 of the apparatus 1000. Each voltage source 218 may be configured to program a group of the electrodes 22 on the chip 10 of the apparatus 1000. For example, the electrode array 20 may include one million electrodes 22 and the apparatus may include five voltage sources 218 each configured to program a different group of electrodes 22 of the array 20. In some embodiments, all five of the voltage sources 218 may be located on the chip 10. In some embodiments, the chip 10 may include a single voltage source 218 configured to program all of the electrodes 22 on the chip 10. In some embodiments, the chip 10 may include wiring (e.g., thin-film wiring lines) configured to connect the current injectors 200 on the chip 10 to contact pads (not shown) to which are bonded wires that lead to one or more off-chip voltage source (not shown), thus permitting the current injectors 200 to be programmed by the off-chip voltage source(s). In some embodiments, the chip 10 may not comprise any voltage source. That is, the voltage source 218 of the apparatus 1000 may be located off-chip. For example, the chip 10 may be supported by a PCB that may also support at least one off-chip voltage source wire-bonded to contact pads on the chip 10. As will be appreciated, other schemes may be used to connect the chip 10 with an off-chip voltage source. In some embodiments, the current injectors 200-1, 200-2, . . . , 200-N may be programmed in a high-throughput process by rastering to address each of the current injectors 200-1, 200-2, . . . , 200-N individually so that custom voltages, which may differ from each other, may be stored in the capacitors 204-1, 204-2, . . . , 204-N.

A computer controller (e.g., the host computer 90) may control the rastering from a first one of the current injectors 200-1, 200-2, . . . , 200-N to a last one of the current injectors 200-1, 200-2, . . . , 200-N. In some embodiments, two or more of the current injectors 200-1, 200-2, . . . , 200-N may be programmed concurrently with a same voltage.

As discussed above, the apparatus 1000 may operate with little or no switching noise caused by clock-controlled switches. For example, in some embodiments of the present technology, prior to commencing a stimulation/sensing process, in which the electrodes 22-1, 22-2, . . . , 22-N are stimulated by stimulation currents, and/or a sensing process, in which OCVs of the electrodes 22-1, 22-2, . . . , 22-N are sensed, the output switches S2, S3 (see FIGS. 2, 3A, 3B) of each of the current injectors 200-1, 200-1, . . . , 200-N may be in an opened state, thus preventing any current generated by the voltage-controlled current generators 206 and the first and second current mirrors 208p, 208n (see FIG. 2) of the current injectors 200-1, 200-2, . . . , 200-N from passing through the output nodes 210-1, 210-2, . . . , 210-N to reach the electrodes 22-1, 22-2, . . . , 22-N. Also, prior to commencing the stimulation/sensing process, the capacitors 204-1, 204-2, . . . , 204-N of the current injectors 200-1, 200-2, . . . , 200-N may be programmed to store voltages that are predetermined to result in desired values for the stimulation currents. The programming may occur by appropriately opening and closing the input switches S1-1, S1-2, . . . , S1-N, which may be controlled using any known technique including techniques that may involve clock signals, i.e., the input switches S1-1, S1-2, . . . , S1-N may be clock-controlled switches. Similarly, the output switches S2, S3 may be controlled using any known technique including techniques that may involve clock signals. As will be appreciated, switching noise that may be generated by the opening and closing of the input switches S1-1, S1-2, . . . , S1-N during programming, and/or by configuring the output switches S2, S3 to be opened, may not be present after the programming is completed, which is before commencing the stimulation/sensing process. When the stimulation/sensing process is to commence, the output switches S2, S3 (see FIGS. 2, 3A, 3B) of each of the current injectors 200-1, 200-1, . . . , 200-N may be controlled to output one of the bidirectional currents produced by the first and second current mirrors 208p, 208n as a stimulation current I_{out_1}, I_{out_2}, . . . , I_{out_N}, by appropriately closing one of the output switches S2, S3, as discussed above. The stimulation/sensing process may then proceed without further opening and/or closing of the output switches S2, S3 until the stimulation/sensing processes ends or until a predetermined time at which a change in direction of the stimulation current is to occur. As will be appreciated, other procedures for operating the apparatus 1000 may be used.

Advantageously, because the current injectors 200-1, 200-1, . . . , 200-N may be employed to produce bidirectional currents with little or no noise resulting from clock-controlled opening and closing of switches, the current injectors 200-1, 200-1, . . . , 200-N may be employed in stimulation/sensing processes where sensed signals may be weak and have a small amplitude and therefore may be difficult to discern from noise that may be detected together with the sensed signals. By minimizing or eliminating clock-generated noise, the current injectors 200-1, 200-1, . . . , 200-N may permit small neurological signals, which otherwise would be difficult to observe due to noise present in conventional multi-electrode apparatuses, to be observable.

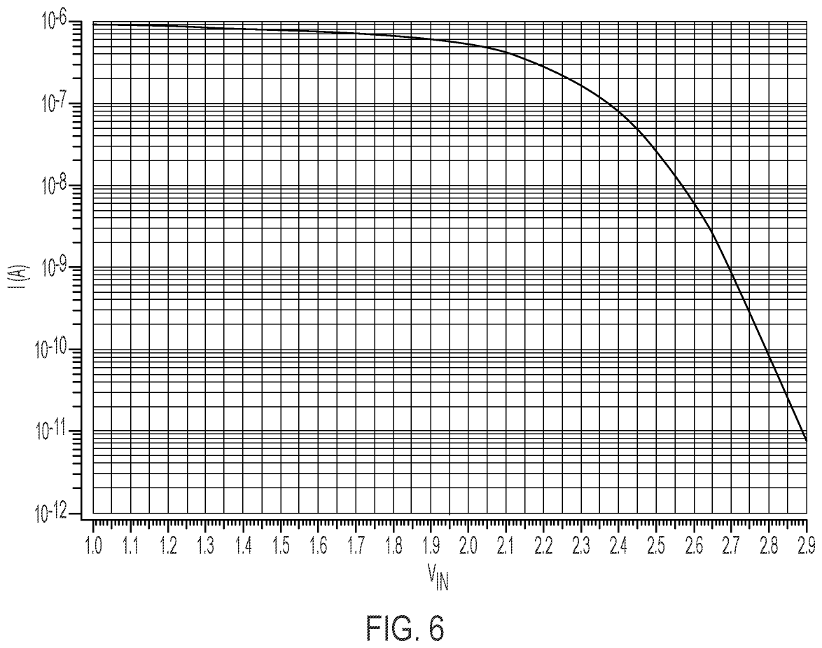

According to some embodiments of the present technology, the current injector 200, 200-1, 200-1, . . . , 200-N may comprise analog components. In some embodiments, the input switch S1, S1-1, S1-2, . . . , S1-N and the output switches S2, S2-1, S2-2, . . . , S2-N, S3, S3-1, S3-2, . . . , S3-N of the current injector 200, 200-1, 200-1, . . . , 200-N may be analog switches, and the capacitor 204, 204-1, 204-2, . . . , 204-N may store an analog voltage having a value that determines an amplitude of a current generated by the current injector 200, 200-1, 200-1, . . . , 200-N. Advantageously, the current injector 200, 200-1, 200-1, . . . , 200-N may be used to generate currents having amplitudes that may vary smoothly over a wide range spanning orders of magnitude. That is, due to the analog nature of the programmed voltage V_{cap}, V_{cap_1}, V_{cap_2}, . . . , V_{cap_N} stored in the capacitor 204, 204-1, 204-2, . . . , 204-N, a current generated from the programmed voltage V_{cap}, V_{cap_1}, V_{cap_2}, . . . , V_{cap_N} need not be restricted to a set of discrete amplitudes but may instead have any amplitude that may be generated from a voltage stored in the capacitor 204, 204-1, 204-2, . . . , 204-N. For example, if the voltage source 218 is able to provide any analog voltage in a predetermined range (e.g., from V_{SS} to V_{DD}) for the programmed voltage V_{cap}, V_{cap_1}, V_{cap_2}, . . . , V_{cap_N}, a desired stimulation current may be set by programming the capacitor 204, 204-1, 204-2, . . . , 204-N to store an appropriate voltage for achieving the desired stimulation current. FIG. 6 shows a chart of current on a logarithmic scale as a function of voltage on a linear scale. As indicated from the chart, a capacitor storing a voltage of about 1.0 V may generate a current of nearly 10-6 A, and a capacitor storing a voltage of about 2.9 V may generate a current of nearly 10-11 A.

Thus, by appropriate selection of an analog voltage for the programmed voltage V_{cap}, V_{cap_1}, V_{cap_2}, . . . , V_{cap_N} Stored in the capacitor 204, 204-1, 204-2, . . . , 204-N, an electrode 22, 22-1, 22-2, . . . , 22-N may be stimulated with a stimulation current Iₒᵤₜ in a wide range of amplitudes spanning several orders of magnitude.

Various embodiments of the present technology may be used to neurological investigations, to stimulate brain regions and to sense signals from the brain regions. In some embodiments, current stimulation may be employed in such investigations for a number of reasons. First, it has been observed that current injectors used to inject current into a cell at a cell-electrode interface may enable sensing and recording of intra-cellular signals through electroporation of the cell's membrane. [J. Abbott et al., “The Design of a CMOS Nanoelectrode Array With 4096 Current-Clamp/Voltage-Clap Amplifiers for Intracellular Recording/Stimulation of Mammalian Neurons,” IEEE Journal of Solid-State Circuits, vol. 55, no. 9, pp. 2567-2582, Sept. 2020.] Second, current injection can also serve as a source of stimulation for the cell and trigger action potentials to fire. For such stimulation, the ability to produce bidirectional currents, i.e., a positive current and a negative current, and provide one or the other selectively and at a wide range of amplitudes, which is possible with the technology disclosed herein, is advantageous. As noted previously, conventional schemes have used switched capacitors controlled by an external clock source to produce injection currents provided to electrodes of multi-electrode apparatuses. However, as noted above, such schemes may suffer from the presence of high-frequency switching noise, which may require extensive measures to minimize. For example, one such measure employs clock synchronization techniques to minimize noise, which may result in a cumbersome form factor that reduces an amount of chip space available for electrodes. Further, a bio-electronic interface may vary from region to region when an actual biological object is being investigated, which may require current injections having amplitudes that vary to balance the variations of the interface. However, with switched-capacitor-based schemes, current injectors typically share a few clock sources and, consequently, such current injectors can only inject a few levels or amplitudes of current. That is, such current injectors are not independent and cannot be individually configured. Moreover, such current injectors are often binary-weighted current sources that utilize an analog-to-digital converter (DAC) to store weight information. However, a drawback of using a binary-weighted current source is that it is not suitable for efficient, high-throughput applications. First of all, to include a DAC in each pixel on a chip would be costly, especially for multi-electrode apparatuses comprised of thousands of pixels or channels each including an electrode, and uses up space on the chip that could otherwise be used for more electrodes. For example, in addition to the increased costs for including the DACs on the chip, for an 8-bit binary-weighted current source, a physical size of each binary-weighted current branch typically scales up exponentially, thus using up a large layout area of the chip. [S. Kotabagi et al., “An 8 Bit Binary Weighted CMOS Current Steering DAC Using UMC 180 nm Technology,” 2020 IEEE 17th India Council International Conference (INDICON), 2020, pp. 1-5.] Secondly, a binary-weighted current source can provide a current magnitude that is 1×, 2×, 4×, 8×, . . . , or 128× that of a generated current, and does not allow for a continuous spectrum of possible currents. Therefore, binary-weighted current sources may not offer stimulation currents that are sufficiently precise enough to modulate bio-electronic interfaces.

In contrast, various embodiments of the DCI described herein may overcome the above-noted drawbacks of switched-capacitor-based schemes and also may overcome the above-noted deficiencies of binary-weighted current sources.

Various embodiments of the DCI disclosed herein may be used for high-throughput in vitro and in vivo electrophysiological investigations, DNA synthesis, biomolecular sensing, and other types of investigations that may benefit from low-noise sensing of signals from multiple electrodes before and/or during and/or after stimulation of the electrodes with individualized currents that may have magnitudes spanning orders of magnitude in range.

Having thus described several aspects and embodiments of the present technology, it is to be appreciated that various alterations, modifications, and improvements will readily occur to those skilled in the art. Such alterations, modifications, and improvements are intended to be part of this disclosure, and are intended to be within the spirit and scope of the invention. Further, though advantages of the present invention are indicated, it should be appreciated that not every embodiment of the technology described herein will include every described advantage. Some embodiments may not implement any feature(s) described as advantageous herein and in some instances one or more of the described features may be implemented to achieve further embodiments. Accordingly, the foregoing description and drawings are by way of example only.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the present invention may be embodied as one or more method(s) in which various embodiments of the structures described above may be used. The acts performed as part of the one or more method(s) may be ordered in any suitable way.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

What is claimed is:

1. A voltage-controlled bi-directional current injector, comprising:
a capacitor configured to store an analog voltage provided by a voltage source;
base-current circuitry configured to receive the analog voltage from the capacitor and to produce a base current from the analog voltage;
a first circuitry branch configured to mirror the base current to produce a first current having a first direction and a magnitude based on the base current;
a second circuitry branch configured to mirror the base current to produce a second current having a second direction and a magnitude based on the base current, the second direction being opposite the first direction; and
output circuitry configured to allow the first current or the second current to be output as an output current,
wherein the analog voltage is provided from the capacitor to the base-current circuitry without use of a clock-signal-controlled switch.

2. The current injector of claim 1, wherein the first and second circuitry branches are arranged in parallel to each other.

3. The current injector of claim 1 or claim 2, wherein:
the base current is provided to the first and second circuitry branches without use a clock-signal-controlled switch between the capacitor and the first circuitry branch and without use of a clock-signal-controlled switch between the capacitor and the second circuitry branch.

4. The current injector of any one of claims 1 through 3, wherein the first and second currents have a same magnitude.

5. The current injector of any one of claims 1 through 4, wherein:
the first circuitry branch includes a first current mirror comprising a plurality of a first type of MOS transistor, and the second circuitry branch includes a second current mirror comprising a plurality of a second type of MOS transistor.

6. The current injector of any one of claims 1 through 5, wherein:
the first circuitry branch includes a plurality of PMOS transistors configured as a first current mirror, and the second circuitry branch includes a plurality of NMOS transistors configured as a second current mirror.

7. The current injector of any one of claims 1 through 6, wherein the first current mirror or the second current mirror is a cascode current mirror.

8. The current injector of any one of claims 1 through 7, wherein each of the first and second current mirrors is a cascode current mirror.

9. The current injector of any one of claims 1 through 8, wherein the output circuitry includes:
a first output switch located on an output side of the first circuitry branch, and a second output switch located on an output side of the second circuitry branch.

10. The current injector of any one of claims 1 through 9, wherein the analog voltage stored by the capacitor is in a range from VSS to VDD.

11. The current injector of any one of claims 1 through 10, wherein VSS is about 0 V and VDD is about 3.3 V.

12. The current injector of any one of claims 1 through 11, wherein an absolute value of the output current is in a range of about 10 pA to about 1 mA.

13. The current injector of any one of claims 1 through 12, wherein the absolute value of the output current is in a range of about 10 pA to about 100 μA.

14. The current injector of any one of claims 1 through 13, wherein the absolute value of the output current is in a range of about 10 pA to about 10 μA.

15. The current injector of any one of claims 1 through 14, further comprising:
an input switch configured to control a connection of the capacitor to the voltage source,
wherein:
the input switch is closed when the capacitor is being addressed by the voltage source to receive the analog voltage, and
the input switch is open when the capacitor is not being addressed by the voltage source.

16. A multi-electrode apparatus, comprising:
a voltage generator;
a plurality of electrodes; and
a plurality of bi-directional current injectors configured to provide output currents to the electrodes, each of the current injectors comprising:
a capacitor configured to store an analog voltage provided by the voltage generator,
base-current circuitry configured to receive the analog voltage from the capacitor and to produce a base current from the analog voltage,
a first circuitry branch configured to mirror the base current to produce a first current having a first direction and a magnitude based on the base current,
a second circuitry branch configured to mirror the base current to produce a second current having a second direction and a magnitude based on the base current, the second direction being opposite the first direction, and
output circuitry configured to allow the first current or the second current to be output as an output current,
wherein, for each of the current injectors, the analog voltage is provided from the capacitor to the base-current circuitry without use of a clock-signal-controlled switch.

17. The apparatus of claim 16, wherein, for each of the current injectors, the first and second circuitry branches are arranged in parallel to each other.

18. The apparatus of claim 16 or claim 17, wherein, for each of the current injectors, the base current is provided to the first and second circuitry branches without use of a clock-signal-controlled switch between the capacitor and the first circuitry branch and without use of a clock-signal-controlled switch between the capacitor and the second circuitry branch.

19. The apparatus of any one of claims 16 through 18, wherein the first and second currents have a same magnitude.

20. The apparatus of any one of claims 16 through 19, wherein, for each of the current injectors:
the first circuitry branch includes a first current mirror comprising a plurality of a first type of MOS transistor, and
the second circuitry branch includes a second current mirror comprising a plurality of a second type of MOS transistor.

21. The apparatus of any one of claims 16 through 20, wherein, for each of the current injectors:
the first circuitry branch includes a plurality of PMOS transistors configured as a first current mirror, and
the second circuitry branch includes a plurality of NMOS transistors configured as a second current mirror.

22. The apparatus of any one of claims 16 through 21, wherein, for each of the current injectors, the first current mirror or the second current mirror is a cascode current mirror.

23. The apparatus of any one of claims 16 through 22, wherein, for each of the current injectors, each of the first and second current mirrors is a cascode current mirror.

24. The apparatus of any one of claims 16 through 23, wherein, for each of the current injectors, the output circuitry includes:
a first output switch located on an output side of the first circuitry branch, and
a second output switch located on an output side of the second circuitry branch.

25. The apparatus of any one of claims 16 through 24, wherein, for each of the current injectors, the analog voltage stored by the capacitor is in a range from VSS to VDD .

26. The apparatus of any one of claims 16 through 25, wherein VSS is about 0 V and VDD is about 3.3 V.

27. The apparatus of any one of claims 16 through 26, wherein, for each of the current injectors, an absolute value of the output current is in a range of about 10 pA to about 1 mA.

28. The apparatus of any one of claims 16 through 27, wherein, for each of the current injectors, the absolute value of the output current is in a range of about 10 pA to about 100 μA.

29. The apparatus of any one of claims 16 through 28, wherein, for each of the current injectors, the absolute value of the output current is in a range of about 10 pA to about 10 μA.

30. The apparatus of any one of claims 16 through 29, wherein the electrodes and the current injectors are disposed on a single substrate.

31. The apparatus of any one of claims 16 through 30, wherein the single substrate is a single semiconductor chip.

32. The apparatus of any one of claims 16 through 31, wherein each of the current injectors is further comprised of an input switch configured to control a connection of the capacitor to the voltage generator, wherein:
the input switch is closed when the capacitor is being addressed by the voltage generator to receive the analog voltage, and
the input switch is open when the capacitor is not being addressed by the voltage generator.

33. The apparatus of any one of claims 16 through 32, wherein the voltage generator is configured to address the current injectors individually to provide individual analog voltages to the current injectors.

34. The apparatus of any one of claims 16 through 33, wherein the individual analog voltages provided by the voltage generator to the current injectors are different from each other.

35. The apparatus of any one of claims 16 through 34, wherein at least two of the current injectors are individually provided with a same analog voltage.

36. The apparatus of any one of claims 16 through 35, further comprising:
a controller configured to control values of the individual analog voltages provided by the voltage generator to the current injectors and to cause the input switches of the current injectors to close individually to receive the individual analog voltages from the voltage generator.

37. The apparatus of any one of claims 16 through 36, wherein the controller is provided with a clock signal to synchronize delivery of the individual analog voltages to the current injectors, such that a value of the analog voltage is known for each of the current injectors.

38. The apparatus of any one of claims 16 through 37, wherein:
the electrodes are arranged in an array of columns and rows, and
the controller causes the input switches of the current injectors to close sequentially from a first one of the electrodes to a last one of the electrodes.

39. The apparatus of any one of claims 16 through 38, wherein a total number of the electrodes is in a range of 2000 to 1,000,000.

40. The apparatus of any one of claims 16 through 39, wherein the total number of the electrodes is in a range of 20,000 to 10,000.

41. The apparatus of any one of claims 16 through 40, wherein the total number of the electrodes is in a range of 10,000 to 100,000.

42. The apparatus of any one of claims 16 through 41, wherein the total number of the electrodes is in a range of 100,000 to 500,000.

43. The apparatus of any one of claims 16 through 42, wherein the total number of the electrodes is in a range of 500,000 to 1,00,000.

44. The apparatus of any one of claims 16 through 43, wherein the electrodes comprise some of or all of a microelectrode array (MEA) and are configured to perform sensing and/or stimulation of an object.

45. A method of using a voltage-controlled bi-directional current injector, comprising:
storing, in a capacitor, an analog voltage provided by a voltage source;
providing, by the capacitor, the analog voltage to base-current circuitry to produce a base current from the analog voltage;
producing, by a first circuitry branch, a first current from the base current, the first current having a first direction and a magnitude based on the base current;
producing, by a second circuitry branch, a second current from the base current, the second current having a second direction and a magnitude based on the base current, the second direction being opposite the first direction; and
outputting the first current or the second current as an output current,
wherein the analog voltage is provided from the capacitor to the base-current circuitry without use of a clock-signal-controlled switch.

46. The method of claim 45, wherein the first and second currents have a same magnitude.

47. The method of claim 45 or claim 46, wherein:
the first circuitry branch includes a first current mirror comprising a plurality of a first type of MOS transistor, and
the second circuitry branch includes a second current mirror comprising a plurality of a second type of MOS transistor.

48. The method of any one of claims 45 through 47, wherein:
the first circuitry branch includes a plurality of PMOS transistors configured as a first current mirror, and
the second circuitry branch includes a plurality of NMOS transistors configured as a second current mirror.

49. The method of any one of claims 45 through 48, wherein the first current mirror or the second current mirror is a cascode current mirror.

50. The method of any one of claims 45 through 49, wherein each of the first and second current mirrors is a cascode current mirror.

51. A method of using a multi-electrode apparatus, comprising:
providing, by a voltage generator, a plurality of analog voltages to a plurality bi-directional current injectors;
at each of the current injectors:
storing, in a capacitor of the current injector, an analog voltage received from the voltage generator,
providing the analog voltage to base-current circuitry of the current injector to produce a base current from the analog voltage,
providing the base current to first and second circuitry branches of the current injector,
producing, by the first circuitry branch, a first current having a first direction and a magnitude based on the base current,
producing, by the second circuitry branch, a second current having a second direction and a magnitude based on the base current, the second direction being opposite the first direction, and
outputting the first current or the second current as an output current of the current injector; and
providing the output currents from the current injectors to a plurality of electrodes,
wherein, at each of the current injectors, the analog voltage is provided from the capacitor to the base-current circuitry without use of a clock-signal-controlled switch.

52. The method of claim 51, wherein the first and second currents have a same magnitude.

53. The method of claim 51 or claim 52, wherein the providing of the analog voltages by the voltage generator comprises addressing the current injectors individually.

54. The method of any one of claims 51 through 53, wherein, at each of the current injectors, the providing of the base current to the first and second circuitry branches occurs without use of a clock-signal-controlled switch between the capacitor and the first circuitry branch and without use of a clock-signal-controlled switch between the capacitor and the second circuitry branch.

55. The method of any one of claims 51 through 54, wherein the providing of the analog voltages by the voltage generator comprises:
controlling the voltage generator to generate the analog voltages to have different values for different ones of the current injectors, and
controlling input switches of the current injectors to open individually such that, for each of the current injectors, a value of the analog voltage provided to the current injector is predetermined for the current injector.

56. The method of any one of claims 51 through 55, wherein the analog voltages provided by the voltage generator to the current injectors are different from each other.

57. The method of any one of claims 51 through 56, wherein at least two of the current injectors are individually provided with a same analog voltage.

58. The method of any one of claims 51 through 57, wherein the providing of the analog voltages by the voltage generator comprises controlling the input switches of the current injectors to open sequentially.

59. The method of any one of claims 51 through 58, wherein:
the first circuitry branch includes a plurality of transistors configured as a first current mirror, and
the second circuitry branch includes a plurality of transistors configured as a second current mirror.

60. The method of any one of claims 51 through 59, wherein the first current mirror or the second current mirror is a cascode current mirror.

61. The method of any one of claims 51 through 60, wherein each of the first and second current mirrors is a cascode current mirror.
